(19) United States
(12) Patent Application Publication
(10) Pub. No.: US 2020/0317976 A1
(43) Pub. Date: Oct. 8, 2020

(54) NOVEL PHASE CHANGE MATERIAL AND METHODS OF USE

(71) Applicant: MONASH UNIVERSITY, Clayton (AU)

(72) Inventors: Douglas MacFARLANE, Clayton (AU); Vijayaraghavan RANGANATHAN, Clayton (AU); Mega KAR, Clayton (AU)

(21) Appl. No.: 16/303,433

(22) PCT Filed: May 18, 2017
(86) PCT No.: PCT/AU2017/000114

§ 371 Date: Nov. 20, 2018

(30) Foreign Application Priority Data
May 20, 2016 (AU) 2016901902

(51) Int. Cl. C09K 5/06; C07D 231/12; C07C 211/63; C07D 233/58; C07D 473/12; C07F 9/54; C07C 309/04

(52) U.S. Cl. CPC C09K 5/063; C07D 231/12; C07C 211/63; C07C 309/04; C07D 473/12; C07F 9/5407; C07D 233/58

(57) ABSTRACT

The invention relates to a phase change material including one or more salts of low vapour pressure and low flammability and an energy storage system, method and device comprising the phase change material.

### FIELD OF INVENTION

The present invention relates to the field of energy storage. In one form, the invention relates to a phase change material for energy storage. In one particular aspect the present invention is suitable for use for storage of thermal energy, including energy derived from solar, geothermal, wind, tidal movement or conventional energy sources.

### BACKGROUND ART

It is to be appreciated that any discussion of documents, devices, acts or knowledge in this specification is included to explain the context of the present invention. Further, the discussion throughout this specification comes about due to the realisation of the inventor and/or the identification of certain related art problems by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

Phase-change materials (PCMs) store and release thermal energy as they change phase. The phase transition most commonly used is the solid to liquid transition (also known as the melting, or fusion, transition). When such PCMs solidify, they release large amounts of energy in the form of the release of the latent heat of fusion, also known as the energy of crystallisation. Conversely when PCMs undergo melting, the latent heat of fusion is absorbed from the immediate environment. For the majority of applications PCMs are encapsulated in sealed containers. PCMs are well known in a variety of contexts from low temperature (−20° C.) applications to high temperature thermal energy storage (>300° C.).

In recent years, interest in the use of PCMs has gained momentum for thermal storage applications in the fields of energy conservation and renewable energy. These PCMs typically have one or more phase transitions with a high enthalpy change at the transition temperature. Solid-liquid phase change materials have proven to be versatile and economically attractive for a number of energy storage applications. For example, International patent application WO 2011/110237 (Siemens AG) describes an energy handling system comprising an energy storage device comprising PCMs for absorbing and temporarily storing thermal energy that has been provided by an energy source (such as wind, tidal or solar sources) and a heat extraction element for extracting thermal energy from the PCM.

In a typical example of their application, PCMs have been used in tanks for storing thermal energy for hot water systems. A typical PCM device of this type is described in an Australian patent application 2011229699 (General Electric Co). PCMs have also been used in solar hot water systems such as those marketed by Cool Air Australia Corporation. The PCM absorbs energy upon its solid-liquid phase change during the day and the latent heat stored in the PCM material maintains the temperature of stored water for longer periods and can be used to pre-heat cold inlet water during the absence of solar energy. Another typical use of PCMs is in heat pipe passive cooling units for direct air cooling applications.

For example, it has been reported that blends of paraffins and fatty acids can be used as latent heat storage components in form-stable phase change materials (hereafter referred to as PCMs). (Kenisarin & Kenisarina, Renewable Sustainable Energy Rev. 2012, 16, 1999-2040). These materials offer a low volume change at the phase transition in contrast to traditional solid-liquid phase-change materials. However, the success of a compound in a PCM application relies on a set of desirable thermophysical properties that includes a high heat of fusion (□H_{f}), a phase transition temperature suited to the application, high thermal conductivity, no tendency to phase separate, good phase-change kinetics (little supercooling, sufficient crystallization rate), high chemical stability, low toxicity, and low flammability.

PCMs can be classified as organic, inorganic, or eutectic materials. In the literature there have been a number of systematic studies of the properties of potential inorganic and organic PCMs. For example, the □H_{f} of organic paraffins has been shown to increase with carbon chain length, varying from the C₁₄ paraffin, which melts at 5.5° C. with □H_{f}=228 J·g⁻¹, to the C34 paraffin, which melts at 75.9° C. with □H_{f}=269 J·g⁻¹. (Sharma et al, Renewable Sustainable Energy Rev. 2009, 13, 318-345).

These enthalpy changes are substantial and useful for a number of low-temperature applications (for example, domestic solar heating) but the low melting point of the materials limits higher-temperature concentrated solar-thermal applications. The heats of fusion of various alcohols, fatty acids, and esters, which offer a range of higher melting points, have also been studied. (Kenisarin & Kenisarina Renewable Sustainable Energy Rev. 2012, 16, 1999-2040; Raemy & Schweizer, J. Therm. Anal. 1983, 28, 95-108).

The sugar alcohols possess high □H_{f} values (>200 J·g⁻¹) with melting transitions in the 100-200° C. range, but they often supercool, which limits their application. Also, organic PCMs are generally volatile and flammable and can cause considerable safety concerns in large scale energy-storage applications. Their thermal conductivities are also generally low. Inorganic PCMs such as salt hydrates. (Farid et al, Energy Conyers. Manage. 2004, 45, 1597-1615) also possess high heats of fusion, in the range of 80-250 J·g⁻¹. For example, KF.4H₂O has □H_{f}=231 J·g⁻¹ at the melting temperature of 20° C., which makes it suitable for thermal energy storage in human comfort applications.

However, these hydrated PCMs often suffer from supercooling and phase separation, which can affect their thermal-storage capacity. Additionally, inorganic PCMs are in many cases corrosive. There have also been reports of organic and inorganic eutectic materials as potential energy-storage materials; for instance, a mixture of trimethylolethane (38.5 wt %), water (31.5 wt %), and urea (30 wt %) has a □H_{f} of 160 J·g⁻¹ with a melting point of 14.4° C. however, this transition point is too low for most applications.

Other materials of interest as thermal-storage media are ionic liquids (ILs), which are molten organic salts and have the added advantages of typically being chemically stable, relatively non-volatile and non-flammable. Hence they are intrinsically more safe in use. Accordingly, studies have been carried out into the phase-change properties of a number of organic salts that are emerging from the ionic liquid field, including protic organic salts. (Vijayraghavan et al, Energy Technol. 2013, 1, 609-612) Protic ionic liquids (PILs) are formed by simple proton transfer from a Bronsted acid to a Bronsted base. Enhancement of the thermal stability of PILs with increasing acid strength is related to the effect of temperature on the proton-transfer equilibrium hence strong bases such as guanidine (Gdm) are particularly attractive for synthesising PILs.

Prior art PCMs typically focus on the choice of materials based on their latent heat of fusion per unit of mass (as expressed in J/g). However, for many practical applications the quantity of energy stored per unit volume (ΔH_{f}^{V}) is the most critical. The temperature of the phase transition also needs to closely match the ideal working temperature for the intended use of the stored energy.

### SUMMARY OF INVENTION

An object of the present invention is to provide improved phase change materials (PCMs).

A further object of the present invention is to alleviate at least one disadvantage associated with the related art.

It is an object of the embodiments described herein to overcome or alleviate at least one of the above noted drawbacks of related art systems or to at least provide a useful alternative to related art systems.

In a first aspect of embodiment described herein there is provided a PCM including one or more salts of low vapour pressure and low flammability, the salt comprising:

(i) a conjugate base chosen from the group comprising benzoate, dihydrogen borate, bromide, tetra-phenylborate, ethanesulphonate, methanesulphonate, phosphonate, phosphate, diphenylphosphate, tosylate, triflate and salicylate; and
(ii) a conjugate acid chosen from the group comprising pyrazolium, triazolium, dimethylethanolammonium, diethylenediammonium diethylammonium, ethylenediammonium, N,N′-dimethylethylenediamine, diethylenetriamine, tetraphenylphosphonium, 1-alkyl-3-methylimidazolium, dipropylammonium, tris(2-aminoethyl)ammonium, imidazolium, caffeinium, 5-phenyl-1H tetrazolium, sodium, guanadinium.

In a second aspect of embodiments described herein there is provided a phase change material comprising; one or more salts of low vapour pressure and low flammability chosen from the group comprising: pyrrazolium methanesulfonate, dimethylethanolammonium methansulfonate, dipropylammonium phosphonate, tris(2-aminoethyl) ammonium triflate, diethylammonium phosphonate, imidazolium diphenyl phosphate and caffeineium triflate.

In a third aspect of embodiments described herein there is provided a phase change material comprising; one or more salts of low vapour pressure and low flammability chosen from the group comprising: [Pyrazolium][Triflate], [Triazolium][Benzoate], [Triazolium][Borate], [Triazolium][Ethanesulphonate], [Triazolium][Salicylate], [Triazolium][Tosylate], [Triazolium][Triflate], [Imidazolium][Ethanesulphonate], [Imidazolium][Tosylate], [Imidazolium][Triflate], [Imidazolium][Diphenylphosphate], [Dimethylethanolammonium][Methanesulphonate], [Tris(2-aminoethyl)ammonium][Triflate], [Caffenium][Triflate], [Ethylenediammonium][Tosylate], [Diethylenediammonium][Tosylate]₂, [Diethylenediammonium][Ethanesulphonate]₂, [5-phenyl-1H-tetrazolium][Methansulphonate], [Triazolium][Methanesulphonate], [N,N′-Dimethylethylenediamine][Methansulphonate]₂, [Diethylenetriamine][Methanesulphonate]₃, [Diethylenetriamine][Methanesulphonate]₂, [Tetraphenylphosphonium][Methanesulphonate], [Tetraphenylphosphonium][Bromide], [Tetraphenylphosphonium][tetra-Phenylborate]a, [C4 mim][tetra-Phenylborate], [C1 mim][tetra-Phenylborate and [Guanadinium][Methansulphonate].

In a particularly preferred embodiment, the PCM of the present invention further includes one or more nucleating agents. Typically, the proportion of salt to nucleating agent is 0.005 to 5 wt %, preferably 0.01 to 1 wt %.

It is also preferred that the nucleating agent of the PCM is a solid material that is insoluble in the salt when it is molten. This is preferred because it has the effect of causing rapid nucleation and growth of salt when it crystallizes. In a preferred embodiment the nucleating agent is a finely divided inert inorganic compounds such as an inert metal oxide or a form of carbon. In a particularly preferred embodiment the nucleating agent is chosen from the group comprising inert compounds such as TiO₂, SiO₂, Al₂O₃, CaO, or other inert metal oxides, or carbon.

Typically, the nucleation agent is in the form of finely divided particles, preferably nanoparticles. The nucleation agent may have any convenient morphology. In the case of carbon this may include, for example, nanoparticles, nanotubules, graphene or fibres.

Typically, the PCM of the present invention comprises a single salt moiety from the group listed above. Alternatively, the PCM may comprise a mixture of one of these salts in combination with one or more known salts. The purpose of mixing is to obtain the desired latent heat of fusion, melting point and freezing point.

In a particularly preferred embodiment, the salt is a protic salt. Where used herein the term protic salt [BH⁺][A⁻] is intended to refer to salts formed by proton transfer from a Bronsted acid AH, to a Bronsted base B according to equation 1 to form the corresponding conjugate acid [A−] and conjugate base [BH+]:

AH+B=[BH⁺][A⁻] (Eqn 1).

In contradistinction, aprotic salts have substituents other than a proton at the site of the labile proton in an analogous protic salt.

The protic salts that are the subject of this invention commonly form at the 1:1 ratio by mole however, it is possible that useful mixtures can be formed at other stoichiometries. For example, it is known that some protic salts advantageously form at the molar ratio of 2:1 (acid:base) and 3:1 (acid:base). Mixtures intermediate between these are possible and may be advantageously used.

The volumetric latent heat of fusion ΔH_{f}^{V}, expresses the quantity of energy that can be absorbed by the material per unit volume of PCM and is preferably large. The molecular features that promote the large latent heat of fusion include one or more of the following:
hydrogen bonding in the solid state that must be disrupted when forming the liquid state for example between N—H moieties and oxyacid moieties;
long chain hydrocarbon structures (—(CH₂-)x that must be disrupted when forming the liquid state; and/or
highly organized arrangements of closely spaced positive and negative charges in the solid state that must be disrupted when forming the liquid state.

Without wishing to be bound by theory, in general, molar volume is also a significant property of PCMs that store a large amount of energy in a small volume of material. This is because at least part of the latent heat of fusion is related to the onset of translational motion of the molecular/ionic species, and thereby the uptake of the kinetic energy of these motions. Accordingly, it is desirable to have as large a number of moles of molecules/ions as possible per unit volume. This requires that the molar volume of the salt be as small as possible.

Preferably, the salt component of the PCM of the present invention has a molar volume in the range 35-200 cm³/mol, preferably 40-150 cm³/mol and more preferably 40-100 cm³/mol.

In essence, embodiments of the present invention stem from the realization that undesirable characteristics (such as flammability and supercooling) exhibited by PCMs of the prior art can at least partially be overcome by the inclusion of one or more salts and one or more nucleating agents.

Supercooling and the reasons why it is undesirable are explained as follows. Supercooling is the phenomenon by which a liquid cools below its equilibrium melting point without freezing because the formation of the solid phase requires nucleation. Nucleation can be slow if (i) the viscosity of the compound is high or (ii) the PCM is a mixture of compounds or (iii) the interfacial free energy or enthalpy between crystal and liquid phases of the PCM is high. Since PCMs preferably have a high enthalpy difference between liquid and crystal, condition (ii) is often true and supercooling results.

The undesirable consequences of supercooling in a PCM include the following:

(i) when heat energy is required form the material, i.e. the heat transfer fluid temperature is below Tm, the temperature of the PCM (T(PCM)) can drop well below Tm before any heat release begins; this creates a “start-up” lag in time before full release of heat is achieved. During this time the heat transfer fluid will not reach its operating temperature (Tₒₚ). Once nucleation occurs, the solid phase will grow, increasing the local temperature towards Tm in the PCM and thereby bringing the heat transfer fluid to its operating temperature;
(ii) the nucleation events are stochastic in time and therefore the start up time lag varies. This creates system unreliability in delivering energy;
(iii) in cases where the temperature of the heat transfer fluid (T_{htf})<Tₒₚ is not optimal for starting up the process, energy is wasted thus lowering the overall system energy efficiency.

For these reasons it is highly desirable that the point at which the PCM begins to freeze during cooling is close to the equilibrium melting point, that is, the degree of supercooling is as small. Accordingly, to limit supercooling, the PCM of the present invention may include at least a nucleating agent to provide a surface on which the nucleation of the solid phase can occur.

Preferably the nucleating agent is combined with the salt in such a manner that nucleation occurs in the bulk of the PCM rather than on the walls of any reservoir or container in which it is stored. This could include, for example, heat transfer fluid piping. If nucleation occurs on the wall of the reservoir, the lowered heat transfer properties of the solid phase of the PCM restricts heat flow from the rest of the PCM, causing T_{htf} to drop below Tₒₚ.

The PCM of the present invention may additionally include minor proportions of other compounds to achieve desirable characteristics. For example, an anti-oxidant may be added to enhance chemical stability. Additives may also be added to enhance thermal conductivity of the material, such as various forms of carbon, including graphene and reduced graphene oxides and metal particles such as metal flakes.

The PCM of the present invention may additionally include gelling agents for the purposes of reducing convective flow or leakage when the material is in its liquid state.

Other aspects and preferred forms are disclosed in the specification and/or defined in the appended claims, forming a part of the description of the invention.

Advantages provided by the present invention comprise the following:
provides increased safety in use by lowering the potential for the material to catch fire,
provides new, economically viable compositions for energy storage applications, and
utilises compounds of high chemical stability, low toxicity and low flammability.

Applications of the present invention comprise the following:
storage of solar-thermal energy for domestic or commercial applications of later use in powering a generator to produce electricity and/or hot water heating and/or space heating and cooling,
storage of thermal energy in large scale industrial installations such as solar power plants, and
use in commercial cooling applications such as heat pipe cooling units for direct air cooling, and
use in conditioning of buildings such as ice-storage facilities

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

### DETAILED DESCRIPTION

Preferred salts for use in the PCMs according to the present invention are listed in Table 1:

TABLE 1
Thermodynamic characteristics of novel organic salts suitable as phase change materials.

| | Tₘ | ΔH_{f} | ΔH_{f}^{v} |
|---|---|---|---|
| Material | (° C. ± 2° C.) | (J/g ± 5%) | (J/cm³± 5%) |
| [Pyrazolium][Methansulphonate] | 164 | 145 | 204 |
| [Dimethylethanolammonium] | 109 | 134 | 186 |
| [Methansulphonate] | | | |
| [Dipropylammonium][Phosphonate] | 138 | 135 | 171 |
| [Tris(2-aminoethyl)ammonium][Tritiate] | 123 | 112 | 168 |
| [Diethylammonium][Phosphonate] | 125 | 107 | 137 |
| [Imidazolium][Diphenylphosphate] | 102 | 93 | 130 |
| [Caffenium][Triflate] | 207 | 76 | 117 |
| [Pyrazolium][Salicylate] | 89 | 131 | |
| [Pyrazolium][Ethanesulphonate] | 115 | 109 | |
| [Pyrazolium][Triflate] | 60, 93, 129* | 13, 17, 13 | |
| [Triazolium][Methanesulphonate] | 135 | 133 | |
| [Triazolium][Benzoate] | 66 | 143 | |
| [Triazolium][dihydrogen Borate] | 120 | 55 | |
| [Triazolium][Ethanesulphonate] | 89 | 98 | |
| [Triazolium][Salicylate] | 67, 93* | 55, 37* | |
| [Triazolium][Tosylate] | 200 | 112 | |
| [Triazolium][Triflate] | 168 | 129 | |
| [Imidazolium][Ethanesulphonate] | 77, 128, 152* | 14, 24, 86* | |
| [Imidazolium][Tosylate] | 70, 111* | 7, 35 | |
| [Imidazolium][Triflate] | 32, 104, 161, 190* | 26, 4, 5, 31* | |
| [Ethylenediammonium][Tosylate] | 120 | 93 | |
| [Diethylenediammonium][Tosylate]₂ | 134 | 21 | |
| [Diethylenediammonium] | 128 | 66 | |
| [Ethanesulphonate]₂ | | | |
| [5-phenyl-1H-tetrazolium] | 115 | 50 | |
| [Methansulphonate] | | | |
| [N,N′-Dimethylethylenediamine] | 87 | 67 | |
| [Methansulphonate]₂ | | | |
| [Diethylenetriamine][Methanesulphonate]₃ | 182 | 90 | |
| [Diethylenetriamine][Methanesulphonate]₂ | 102 | 77 | |
| [Tetraphenylphosphonium] | 313 | 97 | |
| [Methanesulphonate]^{a} | | | |
| [Tetraphenylphosphonium][Bromide]^{a} | 149 | 100 | |
| [Tetraphenylphosphonium][tetra-Phenylborate]^{a} | 201 | 66 | |
| [C4 mim][tetra-Phenylborate]^{a} | 128 | 61 | |
| [C1 mim][tetra-Phenylborate]^{a} | 257 | 116 | |
| [Sodium][methanesulphonate] & | 187 | 76 | |
| [Guanidinium][methansulphonate] | | | |
| [Pyrazolium][Methanesulphonate] & | 132 | 69 | |
| [Guanadinium][Methanesulphonate] | | | |

Cn mim = 1-alkyl-3-methylimidazolium (where n = 1 (methyl), 4 (butyl))
^{a}= aprotic salt
*represents compounds that possess multiple solid-solid transition temperatures and a single melt (in column 2) along with the respective enthalpy of transitions and enthalpy of melt (column 3). For instance, in the case of [Imidazolium][Ethanesulphonate], there are two solid-solid transition temperatures (77° C., 128° C.) before the actual melting occurs at 152° C. The respective enthalpies of transitions are given as 14 and 24 J/g while the enthalpy of melting is 86 J/g. The other compounds in this category should be interpreted in the aforedescribed manner.

This can be compared with typical salts of the prior art such as those listed in the prior art such as Vijayraghavan et al, Energy Tech. 2013, 1 609-612.

It is noted that some of the compounds listed in Table 1 have been disclosed in the prior art but have not hitherto been recognised as a phase change material. These include for example, tetraphenylphosphonium bromide, tetraphenylphosphonium tetra-phenylborate, C4-mim tetra-phenylborate, C1-mim tetra-phenylborate and imidazolium triflate. The remaining compounds listed in Table 1 have not hitherto been described or recognised as a PCM.

The salts as exemplified in Table 1 are pure salts or pure zwitterions. By virtue of their molecular structure these salts absorb large amounts of heat as they melt and release this heat when the subsequently freeze again during cooling. ΔH_{f} is the latent heat of fusion, which expresses the quantity of energy that can be absorbed by the material per unit of PCM.

The salts of the present invention may be used as a pure compound or as a mixture with each other, or with other compounds such that the mixture exhibits a latent heat of fusion (ΔH_{f}) of 70-350 J/cm³/unit of volume, and a melting point of 25-250° C., more preferably 85-200° C., or even more preferably 85-140° C. For example, sodium methanesulphonate does not melt before decomposition (320° C.) and is therefore of no use on its own as a PCM. However, a successful PCM according to the present invention can be created when sodium methanesulphonate is mixed with a compound such as guanidinium methanesuphonate.

Certain advantages may be associated with using a mixture of PCMs, the advantages including the latitude to alter the heat release temperature range to ensure a best match with the intended use of the stored energy. For example, it is possible that two or more PCMs having melting temperatures above the desired temperature range can form a mixture that has a lower melting point, (or liquidus point). Certain combinations of PCMs can melt sharply at what is known as the eutectic temperature.

Additives may also be included in the pure PCM or mixture of PCMs. For example, to avoid supercooling of the PCM liquid before heat release, a non-dissolving component may be added to provide a nucleating function to the mixture. The nucleation agent may be a minor component and could be nano-particulate in form to avoid any separation tendency.

The materials described here can also usefully store energy in solid-solid phase transitions below the melting point where these exist. Either or both phase transition can be of utility as a means of storing thermal energy.

### EXAMPLES

PCMs according to the present invention will be further described with reference to the following non-limiting examples:

### PCMs

### Example 1: Pyrazolium Methanesulphonate

One mole of pyrazole was dissolved in water, neutralised with one mole of methanesulfonic acid and the contents were stirred and kept in an ice bath. The resultant mixture was rotary evaporated at 70° C. (under reduced pressures) to remove water. The compound was then dried under vacuum to remove any residual moisture. The thermal and phase change behaviour of the pyrazolium methanesulfonate formed was studied by differential scanning calorimetry, revealing a melting point at 164° C. with an integrated enthalpy of fusion of 204 J/cm³. This compound was shown to have a very high enthalpy of fusion in the temperature region around 164° C.

### Example 2: Dimethylethanolammonium Methansulfonate

An aqueous solution of 1 mole of 2-dimethylaminoethanol was neutralised with 1 mole of aqueous solution of methanesulfonic acid in an ice bath and the contents were stirred. The water in the mixture was distilled at 70° C. under reduced pressure and the resulting compound was further dried in a vacuum oven to remove traces of moisture. The phase change behaviour of the dimethylethanolammonium methanesulfonate formed was studied by differential scanning calorimetry and obtained a melting point of 109° C. with an integrated enthalpy of fusion of around 186 J/cm³.

### Example 3: Dipropylammonium Phosphonate

One mole of aqueous solution of phosphorous acid was slowly added with stirring to 1 mole of aqueous solution of dipropylamine in an ice bath. The water in the mixture was evaporated to dryness at 70° C. under reduced pressure. The resulting dipropylammonium phosphate was further dried in a vacuum oven and the melting point was determined to be 138° C. with an integrated enthalpy of fusion of around 171 J/cm³.

### Example 4: Tris(2-aminoethyl)ammonium triflate

Tris(2-aminoethyl)ammonium triflate was prepared by neutralising 1 mole of aqueous tris(2-aminoethyl)amine with one mole of aqueous trifluoromethanesulfonic acid in an ice bath. The water in the mixture was removed by distillation at 70° C. under reduced pressures. The resulting tris(2-aminoethyl)ammonium triflate was further dried in a vacuum oven to remove traces of water. The thermal and phase change behaviour was studied by differential scanning calorimetry, revealing a melting point of 123° C. and an integrated enthalpy of fusion of approximately 168 J/cm³.

### Example 5: Diethylammonium Phosphonate

Diethylammonium phosphonate was made by neutralizing 1 mole of aqueous solution of phosphorous acid with 1 mole of aqueous solution of diethylamine in an ice bath. The water in the mixture was removed by distillation at 70° C. under reduced pressure. The diethylammonium phosphonate formed was further dried in a vacuum oven and thermal characterization was carried out by differential scanning calorimetry. The melting point was found to be 125° C. and an integrated enthalpy of fusion to be around 137 J/cm³.

### Example 6: Imidazolium Diphenyl Phosphate

Imidazolium diphenyl phosphate was prepared by melt mixing technique. One mole of imidazole was mixed with one mole of diphenyl phosphate and the mixture was allowed to melt at 100° C. in an oil bath. The homogenous liquid was allowed to cool to room temperature. The solid imidazolium diphenyl phosphate obtained after cooling was analysed by differential scanning calorimetry to investigate the phase change behaviour. The compound begins to crystallise at 36° C., melts at 102° C. and exhibits an integrated enthalpy of fusion of approximately 130 J/cm³.

### Example 7: Caffeineium Triflate

One mole of caffeine was dissolved in hot water, neutralised with one mole of trifluoromethanesulfonic acid and resultant mixture was evaporated to dryness at 70° C. under reduced pressure. The caffeineium triflate formed was further dried in a vacuum oven to remove any residual water. The thermal and phase change behaviour was studied by differential scanning calorimetry, produced a melting point of 207° C. with an integrated enthalpy of fusion of around 117 J/cm³.

### Example 8: Ethylenediammonium Tosylate

The ethylenediammonium tosylate was synthesised by mixing the aqueous solutions of 1 mole of ethylenediamine (EDA) with 1 mole of p-toluenesulfonic acid and distilling off water at 60° C. under reduced pressure using rotatory evaporator. The compound was further dried in a vacuum desiccator at room temperature and analysed by differential scanning calorimetry to investigate the phase change behaviour. The compound melted at around 120° C. and produced an enthalpy of fusion of 93 J/g.

### Example 9: Synthesis of Diethylenediammonium Ditosylate

The diethylenediammonium ditosylate was synthesised using the similar method described above except 2 moles of p-toluenesulfonic acid was used in place of 1 mole of the corresponding acid for making ([EDAH][OTs]. The dried compound was analysed by differential scanning calorimetry and it was found that it melted around 134° C. and exhibited an enthalpy of fusion of 21 J/g.

### Example 10: N,N-Dmethyethylenediammonium dimesylate

The synthesis involve 1 mole of N,N-Dimethylethlenediamine and 2 moles of methanesulfonic acid and the rest of the procedure is the same as described above. The results of differential scanning calorimetry indicate that the compound melted at 87° C. and possessed an enthalpy of fusion of around 67 J/g.

### Example 11: Synthesis of Diethylenetriammonium Dimesylate

The synthesis involves reaction of 1 mole of diethylenetriammonium and 2 moles of methanesulfonic acid and the rest of the procedure is the same as described above. The results of differential scanning calorimetry indicate that the compound melted at 102° C. and possessed an enthalpy of fusion of around 77 J/g.

### Example 12: Diethylenetriammonium Trimesylate

Since this amine is a triamine it was fully protonated by reacting with 3 moles of methanesulfonic acid with 1 mole of the amine and method of synthesis is still the same as described above. The differential scanning calorimetry showed the melting at 182° C. and an enthalpy of fusion of around 90 J/g.

### Example 13: Synthesis of Tetraphenylphosphonium Bromide

Nickel bromide (0.032 moles, 6.9 g), bromobenze (0.064 moles, 10 g) and triphenylphosphine (0.092 moles, 25 g) were mixed in benzonitrile (50 mL). The reaction was refluxed under N₂ for 24 h and then cooled to room temperature. A solution of KBr (10% wt./wt., 150 mL) was added and the organic layer was extracted from dichloromethane (3×75 ml), dried with MgSO₄ and conc. in vacuo to give an off-white solid. Further precipitation of by-product was induced by adding hexane (2×30 mL). The precipitate was filtered and the filtrate was conc. in vacuo to give a white solid (yield: 92%). The differential scanning calorimetry showed the melting at 149° C. and an enthalpy of fusion of 100 J/g.

### Example 14: Tetraphenylphosphonium Tetraphenylborate

Sodium tetraphenylborate (0.005 moles, 1.8 g) and tetraphenylphosphonium bromide (0.0047 moles, 2.0 g) were dissolved in acetone (30 mL) and stirred for 6 h at room temperature. The mixture was then filtered over celite (545), and conc. in vacuo to give white solid (yield: 80%). The differential scanning calorimetry showed the melting at 201° C. and an enthalpy of fusion of around 66 J/g.

The above methods were used to synthesise [C4mim][tetra-Phenylborate] (m.p 128° C., 61 J/g) and [C1mim][tetra-Phenylborate] (m.p 257° C., 116 J/g) where the starting material [C4mim][Br] and [C1mim][Br] were used respectively.

### Example 15: Mixture—Sodium Methanesulfonate & Guanadinium Methanesulfonate

As mentioned previously the PCM of the present invention may comprise a mixture of salts.

In this example a mixture of sodium methanesulfonate and guanadinium methanesulfonate was prepared at 1:1 by mass. The mixture was observed to melt sharply between 187° C. with enthalpy of 75.6 J/g and freeze very sharply at 180° C. The result of mixing has been to lower the melting point usefully, as suitable for lower input temperature applications.

It is noted that sodium methanesulphonate does not melt before decomposition (320° C.) and is therefore of no use on its own as a PCM. However, a successful PCM according to the present invention can be created when sodium methanesulphonate is mixed with guanidinium methanesuphonate.

### Example 16: Mixture—Pyrazolium Methanesulphonate & Guanadinium Methanesulphonate

In this example a mixture of pyrazolium methanesulphonate and guanadinium methanesulphonate was prepared at 1:1 by mass. The mixture produced a broad melting feature consisting of the eutectic melting and a liquidus, between 120 and 160° C. with total enthalpy of melting of 120 J/g. The eutectic melted at 132° C. and had individual enthalpy of fusion of 69 J/g. The eutectic froze very rapidly at 120° C.

### Device

In another aspect of the present invention there is provided an energy storage device for temporarily storing and releasing thermal energy, the storage unit comprising:
a reservoir for containment of the phase change material of the present invention, and
at least one heat transfer means in association with the reservoir.

In one embodiment the heat transfer means is a heat input element and supplies thermal energy to the phase change material. In another embodiment the heat transfer means is a heat extraction element and withdraws thermal energy from the phase change material.

The heat transfer means may be a single device capable of alternatively supplying and extracting thermal energy. Alternatively, two or more separate heat transfer devices may be used. Typically, the supply and extraction of heat is achieved by use of a heat transfer fluid that circulates between the heat transfer means and externally attached components of the device.

### System

The composition of the present invention can be used in any suitable system. For example, in one aspect of the invention there is provided an energy storage system comprising:
(a) a thermal energy source, for example a solar thermal energy source,
(b) a thermal energy storage device including a phase change material according to the present invention for absorbing and temporarily storing the thermal energy, which has been provided by the energy source, and
(c) a thermal energy conversion device which is operatively connected to the thermal energy storage device and which is capable of converting thermal energy into electric energy.

The thermal energy source may for example include one or more of:
a solar thermal collector
an electrical heating element

Thermal energy conversion devices may for example, include one or more of:
an organic Rankine cycle engine which is capable of converting thermal energy into electric energy;
a hot water tank; or
an absorption refrigeration or air conditioning device.

The system is based on the storage and later extraction of thermal energy. The stored thermal energy can be released on demand as needed from the PCM to the energy conversion device for converting the released thermal energy into other forms of energy.

The energy source may be of any convenient type including, solar thermal, geothermal, wind, tidal, photovoltaic or conventional coal power.

The heat extraction element may for example be connected to a heat engine configured for converting thermal energy into mechanical energy. The heat engine may also provide mechanical energy to an electrical generator for conversion into electrical energy. The electrical energy may be supplied to a utility grid.

Typically, the reservoir would comprise a tank such as a steel vessel.

### Method

The present invention also provides a method of energy storage comprising the steps of:
providing thermal energy from an energy source to the phase change composition of the present invention for releasable storage of energy,
releasing at least some of the stored thermal energy from the phase change composition to a heat extraction element in response to an energy need, and
transferring the released thermal energy to an energy conversion device for conversion into electrical energy.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, means-plus-function clauses are intended to cover structures as performing the defined function and not only structural equivalents, but also equivalent structures.

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

1. A phase change material including one or more salts of low vapour pressure and low flammability, wherein the salt comprises:
(i) a conjugate base chosen from the group comprising benzoate, dihydrogen borate, bromide, tetra-phenylborate, ethanesulphonate, methanesulfonate, phosphonate, phosphate, diphenylphosphate, tosylate, triflate and salicylate; and
(ii) a conjugate acid chosen from the group comprising pyrazolium, triazolium, dimethylethanolammonium, diethylenediammonium diethylammonium, ethylenediammonium, N,N′-dimethylethylenediamine, diethylenetriamine, tetraphenylphosphonium, 1-alkyl-3-methylimidazolium, dipropylammonium, tris(2-aminoethyl)ammonium, imidazolium, caffeinium, 5-phenyl-1H tetrazolium, sodium, and guanadinium.

2. The phase change material of claim 1, wherein the one or more salts of low vapour pressure and low flammability are chosen from the group comprising pyrrazolium methanesulfonate, dimethylethanolammonium methansulfonate, dipropylammonium phosphonate, tris(2-aminoethyl) ammonium triflate, diethylammonium phosphonate, imidazolium diphenyl phosphate and caffeineium triflate.

3. The phase change material of claim 1, wherein the one or more salts of low vapour pressure and low flammability are chosen from the group comprising pyrazolium salicylate, pyrazolium ethanesulphonate, pyrazolium triflate, triazolium benzoate, triazolium borate, triazolium ethanesulphonate, triazolium salicylate, triazolium tosylate, triazolium triflate, imidazolium ethanesulphonate, imidazolium tosylate, imidazolium triflate, imidazolium ethanesulphonate, imidazolium tosylate, imidazolium triflate, imidazolium diphenylphosphate, dimethylethanolammonium methanesulphonate, tris(2-aminoethyl)ammonium triflate, caffeinium triflate, ethylenediammonium tosylate, diethylenediammonium(tosylate)₂, diethylenediammonium(ethanesulphonate)₂, and 5-phenyl-1H tetrazolium methanesulphonate.

4. The phase change material of claim 1, wherein the one or more salts of low vapour pressure and low flammability are chosen from the group comprising pyrazolium triflate, triazolium benzoate, triazolium borate, triazolium ethanesulphonate, triazolium salicylate, triazolium tosylate, triazolium triflate, imidazolium ethanesulphonate, imidazolium tosylate, imidazolium diphenylphosphate, dimethylethanolammonium methanesulphonate, tris(2-aminoethyl)ammonium triflate, caffenium triflate, ethylenediammonium tosylate, diethylenediammonium (tosylate)₂, diethylenediammonium(ethanesulphonate)₂, 5-phenyl-1H-tetrazolium methansulphonate, triazolium methanesulphonate, N,N′-dimethylethylenediamine (methansulphonate)₂, diethylenetriamine(methanesulphonate)₃, diethylenetriamine(methanesulphonate)₂, tetraphenylphosphonium methanesulphonate, tetraphenylphosphonium bromide, tetraphenylphosphonium [tetra-phenylborate, C₄mim tetra-phenylborate, C₁mim tetra-phenylborate], and guanadinium methansulphonate.

5. The phase change material of claim 1 further comprising one or more nucleating agents.

6. The phase change material of claim 5, wherein the proportion of salt to nucleating agent is 0.005 to 5 wt %, preferably 0.01 to 1 wt %.

7. The phase change material of claim 6 wherein the nucleating agent is chosen from the group comprising inert inorganic compounds and carbon.

8. An energy storage system comprising:
(a) a thermal energy source,
(b) a thermal energy storage device including a phase change material according to claim 1 for absorbing and temporarily storing thermal energy provided by the energy source, and
(c) a thermal energy conversion device which is operatively connected to the thermal energy storage device and which is capable of converting thermal energy into electric energy.

9. A method of energy storage comprising the steps of:
providing thermal energy from an energy source to a phase change composition according to claim 1 for releasable storage of energy,
releasing at least some of the stored thermal energy from the phase change composition to a heat extraction element in response to an energy need, and
transferring the released thermal energy to an energy conversion device for conversion into electrical energy.

10. An energy storage device for temporarily storing and releasing thermal energy, the storage unit comprising:
a reservoir for containment of the phase change composition of claim 1, and
at least one heat transfer means in association with the reservoir.
